(19) United States
(12) Patent Application Publication
(10) Pub. No.: US 2023/0041175 A1
(43) Pub. Date: Feb. 9, 2023

(54) Ultrasonic Device And Ultrasonic Diagnostic Apparatus

(71) Applicant: SEIKO EPSON CORPORATION, Tokyo (JP)

(72) Inventors: Eiji OSAWA, Matsumoto (JP); Hikaru IWAI, Matsukawa (JP)

(21) Appl. No.: 17/880,718

(22) Filed: Aug. 4, 2022

(30) Foreign Application Priority Data
Aug. 5, 2021 (JP) 2021-128820

(51) Int. Cl. A61B 8/00; B06B 1/06

(52) U.S. Cl. CPC A61B 8/4488; B06B 1/0629; A61B 8/54; B06B 2201/76

(57) ABSTRACT

An ultrasonic device includes a first substrate including a first surface on which a first electrode coupled to a piezoelectric element is placed, a second substrate including a second surface on which a second electrode is placed, an intermediate substrate placed between the first substrate and the second substrate and including a third surface joined to the first surface and a fourth surface facing the second surface, and a bonding portion, wherein the intermediate substrate has a through hole penetrating from the third surface to the fourth surface and a third electrode provided in the through hole and coupled to the first electrode, the second electrode is coupled to the third electrode and electrically coupled to the first electrode via the third electrode, a plurality of the bonding portions are provided in island shapes apart from one another between the second substrate and the intermediate substrate, and at least one of the bonding portions is provided to surround the third electrode.

The present application is based on, and claims priority from JP Application Serial Number 2021-128820, filed Aug. 5, 2021, the disclosure of which is hereby incorporated by reference herein in its entirety.

### BACKGROUND

### 1. Technical Field

The present disclosure relates to an ultrasonic device and an ultrasonic diagnostic apparatus having the ultrasonic device.

### 2. Related Art

In related art, an ultrasonic diagnostic apparatus using an ultrasonic transducer for a probe is known. For example, JP-A-2016-92592 discloses an ultrasonic diagnostic apparatus including an ultrasonic device in which a plurality of ultrasonic transducers are arranged in an array form in a probe.

According to JP-A-2016-92592, a coupling terminal is provided in a front surface as a surface at an exiting side of ultrasonic wave in the ultrasonic device, a flexible board is coupled to the coupling terminal and a pulse signal for driving is supplied via the flexible board. Further, a rigid first reinforcing plate is bonded and fixed to a back surface of the ultrasonic device and increases rigidity of the ultrasonic device. The flexible board for electrical coupling is bended or superimposed and coupled to a connector on the back surface side of the ultrasonic device.

The flexible board is hard to handle and has problems of low implementation yield and difficulty in automation, and replacement by a rigid board is considered. Specifically, it is considered that a coupling pad is provided on the first reinforcing plate on the back surface of the ultrasonic device and the rigid board provided on the rear surface side of the first reinforcing plate is flip-chip-packaged for electrical coupling. In this case, generally, the entire surface is filled with NCP (Non Conductive Paste) such as an epoxy adhesive agent on other portions than the joint portion between an electrode of the rigid board and a coupling bump of the ultrasonic device. However, there is a problem that bonding of the entire surface between the ultrasonic device and the rigid board with NCP affects the characteristics of the ultrasonic transducer. Specifically, the frequency of the ultrasonic wave output by the ultrasonic transducer changes. On the other hand, it is hard to secure rigidity without NCP and the joint portion containing the coupling bump is exposed in the air, and there is a problem that migration due to entry of water may occur.

That is, an ultrasonic device and an ultrasonic diagnostic apparatus using flip-chip packaging and having intended characteristics (including rigidity) are required.

### SUMMARY

An ultrasonic device according to an aspect of the present disclosure includes a first substrate including a first surface on which a piezoelectric element and a first electrode coupled to the piezoelectric element are placed, a second substrate including a second surface on which a second electrode coupled to a control circuit is placed, an intermediate substrate placed between the first substrate and the second substrate and including a third surface joined to the first surface and a fourth surface facing the second surface, and a bonding portion bonding the second substrate and the intermediate substrate, wherein the intermediate substrate has a through hole penetrating from the third surface to the fourth surface and a third electrode provided in the through hole and coupled to the first electrode, the second electrode is coupled to the third electrode and electrically coupled to the first electrode via the third electrode, a plurality of the bonding portions are provided in island shapes apart from one another between the second substrate and the intermediate substrate, and at least one of the bonding portions is provided to surround the third electrode.

An ultrasonic diagnostic apparatus according to an aspect of the present disclosure includes the above described ultrasonic device.

### BRIEF DESCRIPTION OF THE DRAWINGS

FIG. 1 is an appearance diagram of an ultrasonic diagnostic apparatus according to Embodiment 1.

FIG. 2 is a plan view of an ultrasonic device.

FIG. 3 is a sectional view around ultrasonic elements.

FIG. 4 is a sectional view around a coupling pad.

FIG. 5 is a sectional view around a bonding portion.

FIG. 6 is a flowchart of a manufacturing method for the ultrasonic device.

FIG. 7A is a sectional view of one form in a manufacturing process.

FIG. 7B is a sectional view of one form in the manufacturing process.

FIG. 8 is a plan view of an ultrasonic device according to Embodiment 2.

### DESCRIPTION OF EXEMPLARY EMBODIMENTS

### 1. Embodiment 1

Outline of Ultrasonic Diagnostic Apparatus

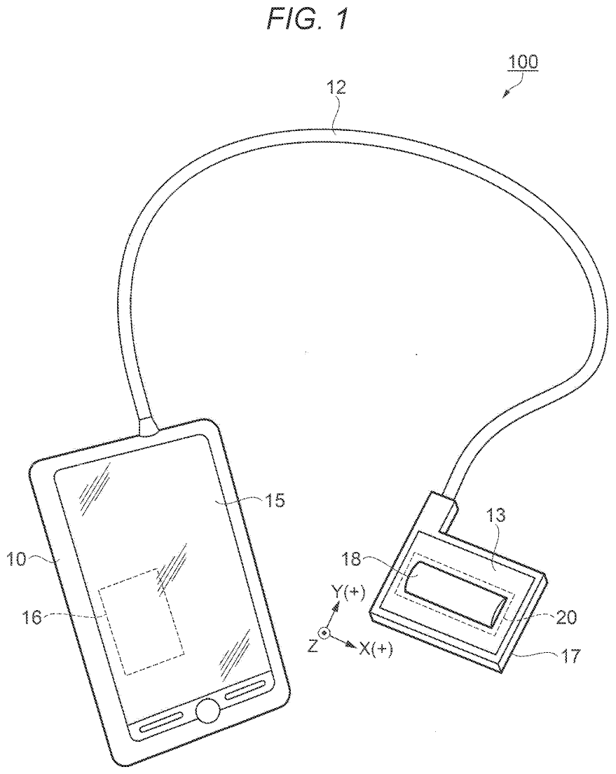

FIG. 1 is a perspective view of an ultrasonic diagnostic apparatus according to Embodiment 1.

First, a schematic configuration of an ultrasonic diagnostic apparatus 100 will be explained using FIG. 1.

The ultrasonic diagnostic apparatus 100 of the embodiment includes a main body 10, a cable 12, a probe 13, etc.

The main body 10 is a main body of the ultrasonic diagnostic apparatus and includes a display unit 15, a control board 16, etc. In a preferable example, the display unit 15 is a liquid crystal panel with touch panel and also functions as an operation unit. The control board 16 is a control unit including a control circuit for controlling transmission and reception of ultrasonic wave by an ultrasonic device 20 provided inside of the probe 13.

The cable 12 is a distribution cable electrically coupling the main body 10 and the probe 13.

The probe 13 is a probe including a case part 17, the ultrasonic device 20, an acoustic lens 18, etc.

The case part 17 has a rectangular box shape and the acoustic lens 18 having a cylindrical lens shape is provided in the surface thereof. The acoustic lens 18 is formed using a material having acoustic impedance close to acoustic impedance in a living body of a subject. In a preferable example, the acoustic lens 18 is formed from silicone resin. Note that, the material is not limited to the silicone resin, but may be a material having acoustic impedance close to that of the living body. Note that a direction along the long side in the rectangular case part 17 is referred to as “X plus direction” and a direction along the short side is referred to as “Y plus direction”.

The ultrasonic device 20 is housed under the acoustic lens 18 in the case part 17. The ultrasonic device 20 is a piezoelectric ultrasonic transducer transmitting and receiving ultrasonic wave. The details of the ultrasonic device 20 will be described later.

When an ultrasonic diagnosis is performed by the ultrasonic diagnostic apparatus 100, scanning is slowly performed with the acoustic lens 18 side of the probe 13 in contact with the living body of the subject. The ultrasonic wave generated in the ultrasonic device 20 is transmitted via the acoustic lens 18 and enters the living body. The ultrasonic wave reflected by the living body is received by the ultrasonic device 20 via the acoustic lens 18.

Then, in the main body 10, an image based on the reception signal of the received ultrasonic wave is generated and a detection result is imaged and displayed on the display unit 15.

### Configuration of Ultrasonic Device

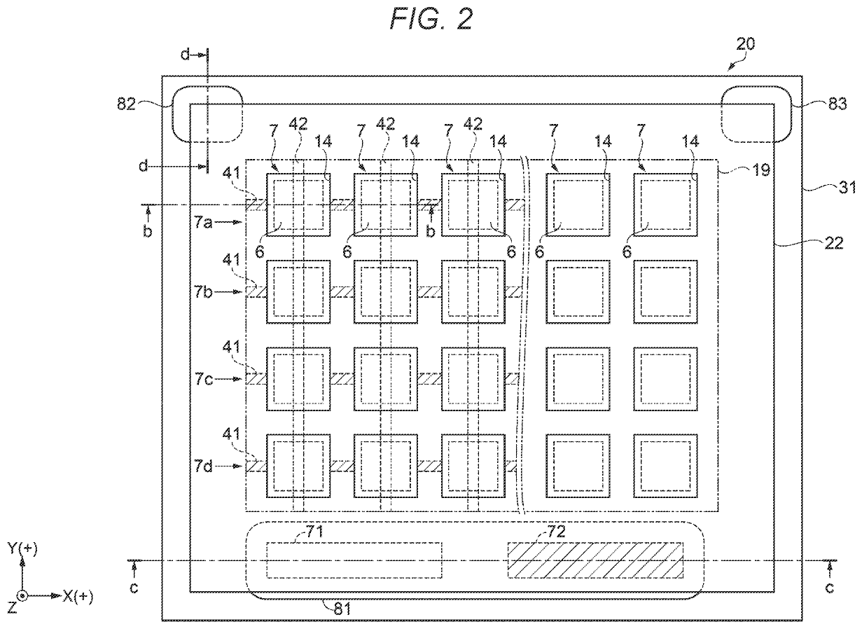

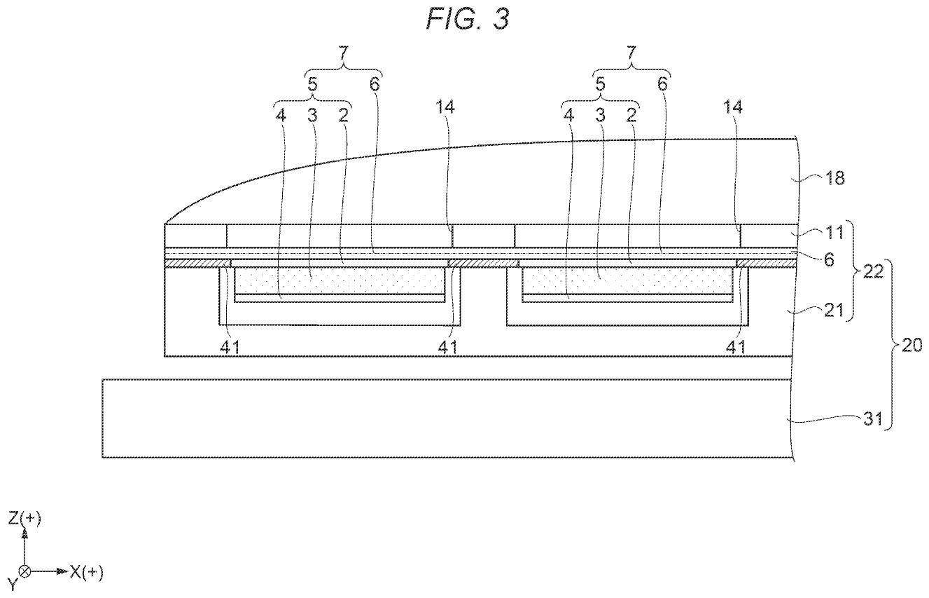

FIG. 2 is a plan view of the ultrasonic device. FIG. 3 is a sectional view along a section b-b in FIG. 2.

As shown in FIG. 2, the ultrasonic device 20 has a rectangular shape in a plan view, and the X plus direction is the long side direction and the Y plus direction is the short side direction. As shown in FIG. 3, the ultrasonic device 20 has a configuration in which an ultrasonic substrate 22 is stacked on a second substrate 31.

Returning to FIG. 2, the ultrasonic substrate 22 has a rectangular shape slightly smaller than the second substrate 31, and an element array area 19 in which a plurality of ultrasonic elements 7 are arranged in an array form is provided at the center thereof.

In FIG. 2, the ultrasonic elements 7 are arranged in four rows in the Y plus direction and five columns in the X plus direction. Note that the arrangement is not limited to the arrangement form, but may be appropriately set according to the size and specifications of the probe 13 (FIG. 1). Further, the row of the ultrasonic elements 7 extending in the Y plus direction is referred to as “element row 7a”, from the element row 7a toward the Y minus direction, the second row is referred to as “element row 7b”, the third row is referred to as “element row 7c”, and the fourth row is referred to as “element row 7d”.

As shown in FIG. 2, one ultrasonic element 7 has a nearly square shape in the plan view and the square is defined by an opening portion 14. Note that, in FIG. 2, the appearance of the element array area 19 is shown at the X plus side of a broken line and wires in the under layer are shown through at the minus side of the broken line. A vibrating film 6 is exposed in the opening portion 14 and the ultrasonic wave generated by the ultrasonic element 7 is transmitted by vibration of the vibrating film 6. Further, the vibrating film 6 also functions as a vibrating film when ultrasonic wave is received.

A coupling bump 71 and a coupling bump 72 are provided side by side in the X plus direction at the Y minus side of the element array area 19. The coupling bump 71 and the coupling bump 72 are coupling terminals for electrical coupling between the ultrasonic substrate 22 and the second substrate 31. Specifically, the bumps are coupling bumps for flip-chip packaging of the ultrasonic substrate 22 on the second substrate 31.

As shown in FIG. 2, the coupling bump 71 and the coupling bump 72 are surrounded by a bonding portion 81. Further, a bonding portion 82 and a bonding portion 83 are provided in two vortex portions at the Y plus side of the ultrasonic substrate 22.

FIG. 3 is the sectional view along the section b-b in FIG. 2 and shows sectional configurations of the ultrasonic elements 7. As shown in FIG. 3, the ultrasonic substrate 22 has a configuration in which a first substrate 11 and an intermediate substrate 21 are stacked.

As the first substrate 11, a silicon substrate is used in a preferable example. Note that the substrate is not limited to the silicon substrate, but may be a hard rigid substrate.

The vibrating film 6 is provided at the Z minus side of the first substrate 11. The vibrating film 6 has a two-layer configuration in a preferable example. Specifically, the first layer of the first substrate 11 at the base material side is a silicon oxide (SiO₂) layer and the second layer is a zirconium oxide (ZrO₂) layer. It is preferable to set the film thickness of the vibrating film 6 based on the resonance frequency of the transmitted and received ultrasonic wave. Note that the film is not limited to the configuration, but may be formed using a hard material that can resonate by the ultrasonic wave in a single layer.

As described above, a plurality of the opening portions 14 are provided in the first substrate 11.

The ultrasonic elements 7 are provided in correspondence with the opening portions 14 and include the vibrating films 6, piezoelectric elements 5, etc.

The piezoelectric element 5 includes an electrode 2, a piezoelectric material 3, an electrode 4, etc.

The electrode 2 provided on the vibrating film 6 is a drive electrode for the piezoelectric material 3 and formed using a conducting material. As the conducting material, e.g. a metal material such as platinum (Pt), iridium (Ir), gold (Au), aluminum (Al), copper (Cu), titanium (Ti), or stainless steel, a tin oxide-containing conducting material such as indium tin oxide (ITO) or fluorine-doped tin oxide (FTO), a zinc oxide-containing conducting material, an oxide conducting material such as strontium ruthenate (SrRuO₃), lanthanum nickel oxide (LaNiO₃), or element-doped strontium titanate, conducting polymer, or the like may be used.

As the piezoelectric material 3 provided on the electrode 2, a piezoelectric material layer of lead zirconate titanate (PZT) is used in a preferable example. Note that the material is not limited to that, but a piezoelectric material having equal displacement may be used.

The electrode 4 provided on the piezoelectric material 3 is a drive electrode for the piezoelectric material 3 and formed from the same conducting material as that of the electrode 2.

The intermediate substrate 21 is a lid member covering not to hinder the vibration in the plurality of ultrasonic elements 7 and a silicon substrate is used in a preferable example. Note that the substrate is not limited to the silicon substrate, but may be a hard rigid substrate. As shown in FIG. 3, a plurality of recessed portions defining the ultrasonic elements 7 are provided in the intermediate substrate 21. The intermediate substrate 21 is bonded and fixed to the vibrating film 6 of the first substrate 11.

In a completion state of the ultrasonic substrate 22 in which the first substrate 11 and the intermediate substrate 21 are joined, a space is formed under the ultrasonic element 7 like the space by the opening portion on the upside. The spaces are provided on the upside and the downside of the ultrasonic element 7, and thereby, the vibration of the ultrasonic element 7 is not hindered.

The electrodes 2 of the ultrasonic elements 7 adjacent in the X plus direction are electrically coupled by first wires 41. In a preferable example, the first wires 41 are formed together in the same process of the formation of the electrodes 2.

As shown in FIG. 2, all of the electrodes 2 in the plurality of ultrasonic elements 7 forming the element row 7a are electrically coupled by the first wire 41. The same applies to the element row 7b, the element row 7c, and the element row 7d.

Further, similarly, in the column direction, all of the electrodes 4 in the ultrasonic elements 7 adjacent in the Y minus direction are electrically coupled by second wires 42. Note that insulating layers are provided between the first wires 41 and the second wires 42 and isolation is secured.

In the embodiment, a driving method of supplying a common drive signal to all ultrasonic elements 7 of the element array area 19 and alternately transmitting and receiving ultrasonic wave in time sequence is employed. Specifically, the common drive signal is supplied to all ultrasonic elements 7 via the coupling bump 71. In a preferable example, the drive signal is a burst-wave drive signal and periodically transmitted with the reception times secured in time sequence, and thereby, transmission and reception of ultrasonic wave are alternately performed. Further, from the coupling bump 72, e.g. a common potential such as a ground potential is supplied to all of the ultrasonic elements 7.

Joint Configuration between Substrates

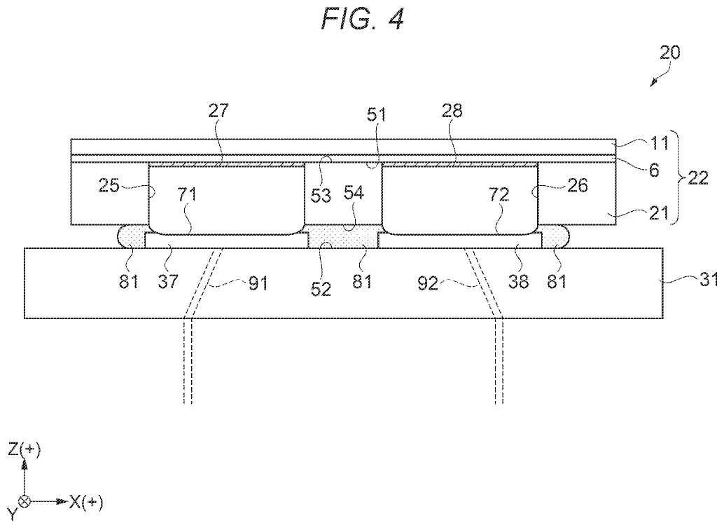

FIG. 4 is a sectional view along a section c-c in FIG. 2.

Next, a joint configuration between the intermediate substrate 21 of the ultrasonic substrate 22 and the second substrate 31 is explained.

As shown in FIG. 4, two through holes 25 and 26 are formed in the intermediate substrate 21. A first electrode 27 is provided in the bottom portion of the through hole 25 in the first substrate 11. Similarly, a first electrode 28 is provided in the bottom portion of the through hole 26. In other words, the first electrode 27 and the first electrode 28 are provided on a first surface 51 as a surface at the Z minus side in the first substrate 11. The first electrode 27 is electrically coupled to the second wires 42 (FIG. 2) by wires (not shown). The first electrode 28 is electrically coupled to the first wires 41 (FIG. 2) by wires (not shown). That is, the first electrodes 27, 28 are electrically coupled to the piezoelectric elements 5 (FIG. 3).

Further, the coupling bump 71 formed using a resin adhesive agent containing metal filler is provided in the through hole 25. Similarly, the coupling bump 72 is provided in the through hole 26. The coupling bumps 71, 72 are coupling bumps having anisotropic conductivity. When the coupling bumps are pressingly fixed to the second substrate 31, electrical coupling may be secured in the Z-axis directions. The coupling bumps 71, 72 correspond to third electrodes.

In a preferable example, as the coupling bumps 71, 72, materials of epoxy region adhesive agents containing silver filler are used. Note that the coupling bumps are not limited to those, but may be materials having equal anisotropic conductivity. As the epoxy region adhesive agent, e.g. a urethane resin or silicone resin adhesive agent may be used. As the metal filler, a metal such as gold, copper, nickel, or tin or a metal oxide such as indium oxide may be used. Not only the metal filler but also conducting filler such as carbon fiber or nanotube may be used.

In the second substrate 31, a second electrode 37 and a second electrode 38 are provided in the positions corresponding to the through hole 25 and the through hole 26. In other words, the second electrodes 37, 38 are provided on a second surface 52 as a surface at the Z plus side of the second substrate 31.

A through wire 91 is coupled to the second electrode 37. The through wire 91 is electrically coupled to the control board 16 (FIG. 1) via a wire extending from the rear surface of the second substrate 31 to the outside and the cable 12 (FIG. 1). Similarly, a through wire 92 is coupled to the second electrode 38, and the through wire 92 is also electrically coupled to the control board 16 (FIG. 1). In other words, the second electrodes 37, 38 are coupled to the control circuit of the control board 16 (FIG. 1).

The second electrode 37 is a metal electrode and has a three-layer configuration of an Ni layer, a Pt layer, and an Au layer in a preferable example. The second electrode 38 has the same configuration. Note that the second electrodes are not limited to those, but may be metals that can electrically couple between the coupling bumps 71, 72 in single layers.

In the intermediate substrate 21, a surface facing the first surface 51 of the first substrate 11 is a third surface 53 and a surface facing the second surface 52 of the second substrate 31 is a fourth surface 54. In other words, the intermediate substrate 21 is placed between the first substrate 11 and the second substrate 31 and includes the third surface 53 joined to the first surface 51 and the fourth surface 54 facing the second surface 52. Further, the intermediate substrate 21 includes the through holes 25, 26 penetrating from the third surface 53 to the fourth surface 54 and the coupling bumps 71, 72 provided in the through holes 25, 26 and coupled to the first electrodes 27, 28. Furthermore, the second electrodes 37, 38 are coupled to the coupling bumps 71, 72 and electrically coupled to the first electrodes 27, 28 via the coupling bumps 71, 72.

As shown in FIG. 4, the bonding portion 81 is placed around the joint portion in which the coupling bump 71 and the second electrode 37 are joined. In a preferable example, the bonding portion 81 uses an epoxy resin adhesive agent as NCP. Note that the bonding portion is not limited to that, but may be an adhesive agent having an insulating property, moisture resistance, and an adhesion property equal to the epoxy resin adhesive agent. For example, a urethane resin or silicone resin adhesive agent may be used.

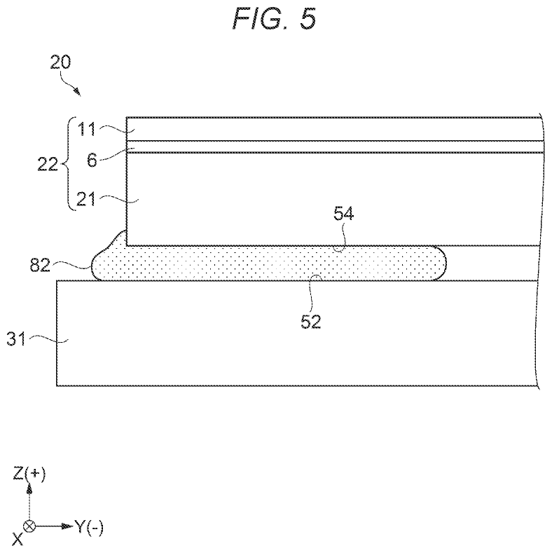

FIG. 5 is a sectional view along a section d-d in FIG. 2.

As shown in FIG. 5, the bonding portion 82 is provided between the intermediate substrate 21 and the second substrate 31 in the vertex portion of the ultrasonic substrate 22. The bonding portion 82 is the same adhesive agent as the bonding portion 81. The bonding portion 82 bonds the second surface 52 of the second substrate 31 and the fourth surface 54 of the intermediate substrate 21. A part of the bonding portion 82 protrudes from the intermediate substrate 21 into contact with the side surface of the peripheral edge. In other words, the part of the bonding portion 82 is also provided in the peripheral edge part of the intermediate substrate 21.

A part in which the bonding portion 82 is not provided between the ultrasonic substrate 22 and the second substrate 31 is an air space. In other words, gaps are provided in parts in which the bonding portion 81 and the bonding portion 82 are not provided between the ultrasonic substrate 22 and the second substrate 31.

Returning to FIG. 2, in the plan view, the coupling bump 71 has the rectangular shape and the long side extends in the X plus direction. The coupling bump 72 adjacent to the coupling bump 71 similarly has the rectangular shape. The second electrode 37 (FIG. 4) overlapping with the coupling bump 71 is also formed in a rectangular shape in the plan view. The second electrode 38 (FIG. 4) overlapping with the coupling bump 72 is formed in the same shape. That is, the coupling bump 71 and the coupling bump 72 are placed side by side along one long side of the ultrasonic substrate 22.

As shown in FIG. 2, the bonding portion 81 is provided in a rectangular shape surrounding the coupling bumps 71, 72 in the plan view. That is, the bonding portion 81 having the rectangular shape is provided along one long side of the ultrasonic substrate 22. The length of the bonding portion 81 in the long side direction is a length equal to or more than a half of the length of the long side of the intermediate substrate 21. The bonding portion 81 is also provided between the coupling bump 71 and the coupling bump 72. Further, a part of the bonding portion 81 protrudes from the long side at the Y minus side of the ultrasonic substrate 22. In other words, the part of the bonding portion 81 is also provided in the peripheral edge of the intermediate substrate 21. On the other hand, the long side at the Y plus side of the bonding portion 81 does not extend over the element array area 19 and the bonding portion 81 is provided apart from the element array area 19.

The bonding portion 82 is provided on one end and the bonding portion 83 is provided on the other end in the other long side of the ultrasonic substrate 22. A part of the bonding portion 82 protrudes from one vertex of the ultrasonic substrate 22. That is, the part of the bonding portion contacts the side surface of the long side and the side surface of the short side in the intermediate substrate 21 of the ultrasonic substrate 22. Similarly, a part of the bonding portion 83 protrudes from the other vertex of the ultrasonic substrate 22. That is, the part of the bonding portion contacts the side surface of the long side and the side surface of the short side in the intermediate substrate 21 of the ultrasonic substrate 22. On the other hand, the bonding portions 82, 83 do not spread over the element array area 19 and the bonding portions 82, 83 are provided apart from the element array area 19. In other words, the bonding portions 81, 82, 83 are provided in parts not overlapping with the element array area 19 in the plan view. A plurality of the bonding portions 81, 82, 83 are provided in island shapes apart from one another between the second substrate 31 and the intermediate substrate 21 and at least one of the bonding portions is provided to surround the coupling bumps 71, 72.

The bonding portion 81, the bonding portion 82, and the bonding portion 83 are placed in a truss arrangement with the element array area 19 in between. Specifically, a triangle with the center of the bonding portion 81 along one long side of the ultrasonic substrate 22 as a vertex and the other long side as a bottom side is assumed, and the bonding portion 82 and the bonding portion 83 are provided on the vertices of the bottom side. Thereby, the joint strength between the ultrasonic substrate 22 and the second substrate 31 is secured without hindrance to the vibration in the element array area 19. In other words, the bonding portions 81, 82, 83 are provided in at least three positions apart in the periphery of the element array area 19. Note that, not limited to the three positions, but the bonding portions may be provided in four or more positions.

### Manufacturing Method for Ultrasonic Device

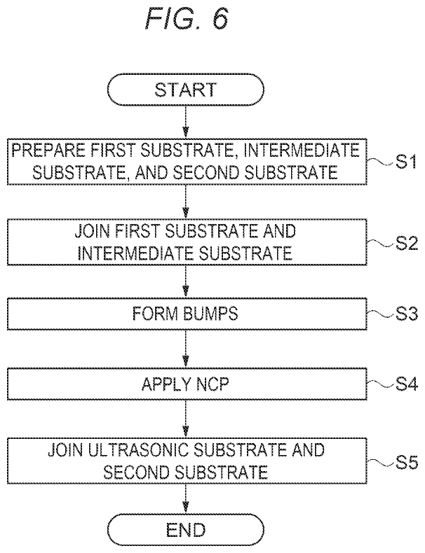

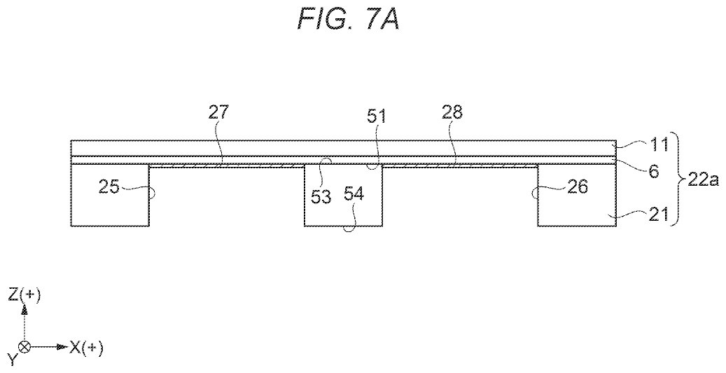

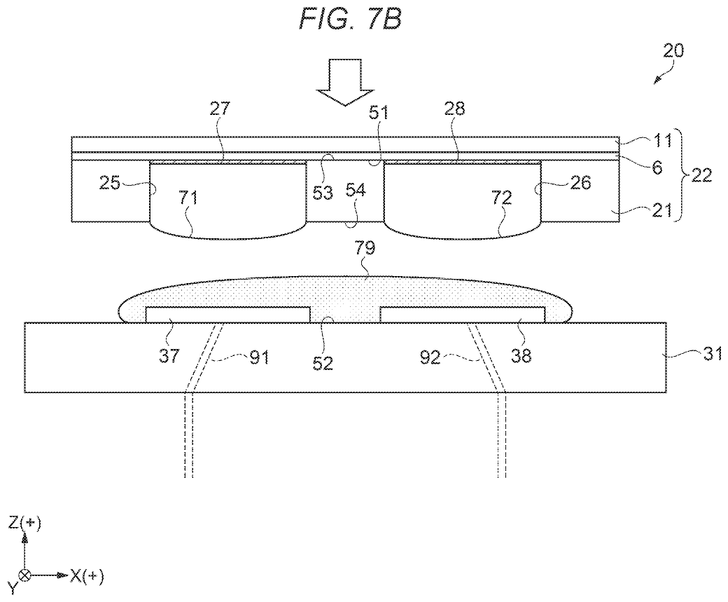

FIG. 6 is a flowchart showing a manufacturing method for the ultrasonic device. FIGS. 7A and 7B are process diagrams along the section c-c in FIG. 2.

Here, the manufacturing method for the ultrasonic device 20 will be explained with reference mainly to FIG. 6 and appropriately to FIGS. 7A, 7B, and 4.

At step S1, the first substrate 11, the intermediate substrate 21, and the second substrate 31 are prepared. Specifically, the first substrate 11, the intermediate substrate 21, and the second substrate 31 respectively manufactured in different processes are prepared.

At step S2, the first substrate 11 and the intermediate substrate 21 are joined. Specifically, the intermediate substrate 21 is bonded to the vibrating film 6 of the first substrate 11. In a preferable example, a silicone adhesive agent is used. FIG. 7A shows an ultrasonic substrate 22a in the manufacturing process in which the first substrate 11 and the intermediate substrate 21 are joined.

At step S3, the coupling bumps 71, 72 are formed. Specifically, the two through holes 25 and 26 of the intermediate substrate 21 are respectively filled with epoxy resin adhesive agents containing silver filler and heated and cured. In a preferable example, the through hole 25 and the through hole 26 are filled with adequate amounts of the epoxy resin adhesive agents using a dispenser. In this regard, as shown in FIG. 7B, the coupling bumps 71, 72 are protruded in fixed amounts from the fourth surface 54. Then, the coupling bumps 71, 72 are formed by curing of the epoxy resin adhesive agents by heating in a constant-temperature reservoir set at a predetermined temperature.

At step S4, the NCP is applied. In a preferable example, as shown in FIG. 7B, an epoxy resin adhesive agent 79 is applied onto the second electrodes 37, 38 of the second substrate 31. In a preferable example, an adequate amount of the epoxy resin adhesive agent 79 is applied onto the second electrodes 37, 38 using a dispenser. Further, in parallel, the epoxy resin adhesive agent is applied to parts to be the bonding portions 82, 83. Note that the epoxy resin adhesive agent may be applied to the ultrasonic substrate 22 side.

A step S5, the ultrasonic substrate 22 and the second substrate 31 are joined. Specifically, as shown in FIG. 7B, predetermined pressure is applied with the ultrasonic substrate 22 on the second substrate 31 and the epoxy resin adhesive agents are cured by heating in a constant-temperature reservoir set at a predetermined temperature. In other words, the ultrasonic substrate 22 is flip-chip-packaged on the second substrate 31.

Thereby, the ultrasonic device 20 in FIG. 4 is completed.

As described above, according to the ultrasonic device 20 and the ultrasonic diagnostic apparatus 100 of the embodiment, the following effects may be obtained.

The ultrasonic device 20 includes the first substrate 11 including the first surface 51 on which the piezoelectric element 5 and the first electrodes 27, 28 coupled to the piezoelectric element 5 are placed, the second substrate 31 including the second surface 52 on which the second electrodes 37, 38 coupled to the control circuit are placed, the intermediate substrate 21 placed between the first substrate 11 and the second substrate 31 and including the third surface 53 joined to the first surface 51 and the fourth surface 54 facing the second surface 52, and the bonding portions 81, 82, 83 bonding the second substrate 31 and the intermediate substrate 21. Further, the intermediate substrate 21 includes the through holes 25, 26 penetrating from the third surface 53 to the fourth surface 54 and the coupling bumps 71, 72 as the third electrodes provided in the through holes 25, 26 and coupled to the first electrodes 27, 28, the second electrodes 37, 38 are coupled to the coupling bumps 71, 72 and electrically coupled to the first electrodes 27, 28 via the coupling bumps 71, 72, the plurality of bonding portions 81, 82, 83 are provided in island shapes apart from one another between the second substrate 31 and the intermediate substrate 21, and at least one of the bonding portions 81, 82, 83 is provided to surround the coupling bumps 71, 72.

According to the configuration, unlike the configuration of related art in which the entire surfaces between the ultrasonic device and the rigid substrate are bonded, the second substrate 31 and the intermediate substrate 21 are joined by the island-shaped bonding portions 81, 82, 83 apart from one another, and thereby, both of the bonding areas become smaller and the influence on the characteristics of the ultrasonic device 20 may be reduced.

Further, the bonding portion 81 is provided to surround the coupling bumps 71, 72, and thereby, entry of water into the joint portions including the coupling bumps 71, 72 may be prevented.

Therefore, the ultrasonic device 20 using flip-chip packaging and having intended characteristics may be provided.

Further, the piezoelectric element 5 is provided in contact with the vibrating film 6, the element array area 19 in which the plurality of piezoelectric elements 5 are regularly arranged is provided, and the bonding portions 81, 82, 83 are provided in parts not overlapping with the element array area 19 in a plan view.

According to the configuration, the bonding portion is not provided in the element array area 19, and thereby, oscillation of ultrasonic wave by the ultrasonic elements 7 is not hindered and the intended characteristics may be obtained.

In the plan view, the intermediate substrate 21 has the rectangular shape, the element array area 19 is provided so that the center of the intermediate substrate 21 is placed inside the element array area 19, and the bonding portions 81, 82, 83 are provided at least in three positions apart in the periphery of the element array area 19.

According to the configuration, the second substrate 31 and the intermediate substrate 21 are fixed by the three or more bonding portions surrounding the element array area 19 in the plan view, and thereby, rigidity of the ultrasonic device 20 as a composite structure may be secured.

Particularly, as shown in FIG. 2, when the substrates are fixed by the bonding portions 81, 82, 83 in the truss arrangement with the element array area 19 in between, rigidity necessary as the structure may be secured even by the fixation in the three positions.

The coupling bumps 71, 72 as the third electrodes are bumps formed using resin adhesives containing metal filler.

According to the configuration, because of the anisotropic conductivity of the coupling bumps 71, 72, electrical coupling between the first electrodes 27, 28, the second electrodes 37, 38 may be reliably established.

The bonding portions 81, 82, 83 are insulating resin adhesive agents.

According to the configuration, entry of water into the joint portions including the coupling bumps 71, 72 may be prevented and electrical isolation may be secured.

A part of the bonding portions 81, 82, 83 is also provided in the peripheral edge part of the intermediate substrate 21.

According to the configuration, the bonding strength to the second substrate 31 may be further increased by the bonding portion protruding to the peripheral edge part of the intermediate substrate 21.

The ultrasonic diagnostic apparatus 100 includes the ultrasonic device 20.

According to the configuration, the ultrasonic diagnostic apparatus 100 using flip-chip packaging and having intended characteristics may be provided.

### Embodiment 2

### Different Circuit Configuration of Ultrasonic Device

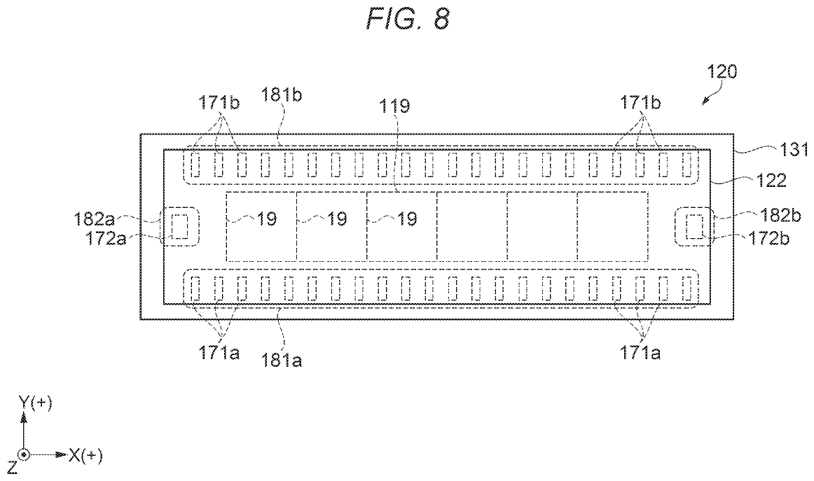

FIG. 8 is a plan view of an ultrasonic device according to the embodiment and corresponds to FIG. 2.

As shown in FIG. 8, an ultrasonic device 120 of the embodiment has a rectangular shape elongated in the X plus direction and includes an element array area 119 at the center thereof. The element array area 119 has a multi-channel configuration in which six of the element array areas 19 of Embodiment 1 are arranged in the X plus direction. The ultrasonic device 120 has a configuration in which an ultrasonic substrate 122 is flip-chip-packaged on a second substrate 131. The ultrasonic substrate 122 has a rectangular shape slightly smaller than the second substrate 131. In correspondence with the multiple channels, a plurality of coupling bumps 171a to which drive signals are input are provided at the Y minus side of the element array area 119. Similarly, a plurality of coupling bumps 171b are provided at the Y plus side of the element array area 119. Note that the coupling bumps 171a, 171b also function as receiving terminals of reception signals of ultrasonic wave received by the ultrasonic elements 7.

Further, coupling bumps 172a, 172b to which a common potential is supplied are provided substantially in the middle of the two short sides of the ultrasonic substrate 122. Furthermore, a bonding portion 181a surrounding the plurality of coupling bump 171a is provided along one long side of the ultrasonic substrate 122. Similarly, a bonding portion 181b surrounding the plurality of coupling bump 171b is provided along the other long side.

A bonding portion 182a is provided around the coupling bump 172a and a bonding portion 182b is provided around the coupling bump 172b. The other configurations are the same as those of Embodiment 1. As below, the same configuration parts as those of Embodiment 1 will have the same signs and the overlapping explanation will be omitted.

The bonding portions 181a, 181b and the bonding portions 182a, 182b are placed respectively independently in island shapes outside of the element array area 119.

The bonding portions 181a, 181b are provided along the long side of the ultrasonic substrate 122 and a part of the bonding portions protrudes out of the ultrasonic substrate 122. Similarly, the bonding portions 182a, 182b are provided along the short side of the ultrasonic substrate 122 and a part of the bonding portions protrudes out of the ultrasonic substrate 122.

In the ultrasonic device 120 having the above described configuration, for example, the element array areas 19 in the element array area 119 may be alternatively provided exclusively for transmission and reception. Or, the odd-numbered element rows can be exclusively for transmission and the even-numbered element rows can be exclusively for reception. Or, various types of driving may be performed such that switching elements are provided with respect to each of the ultrasonic elements 7 and the ultrasonic elements 7 are individually actively driven.

As described above, according to the ultrasonic device 120 of the embodiment, the following effects may be obtained in addition to the effects of Embodiment 1.

In the ultrasonic device 120, the bonding portions 181a, 181b, 182a, 182b are provided in island shapes apart from one another between the second substrate 131 and the ultrasonic substrate 122, and the respective bonding portions are provided to surround the corresponding coupling bumps.

According to the configuration, the second substrate 131 and the ultrasonic substrate 122 are joined by the bonding portions 181a, 181b, 182a, 182b apart in the island shapes, and thereby, both of the bonding areas become smaller and the influence on the characteristics of the ultrasonic device 20 may be reduced. Further, the bonding portions are provided to surround the corresponding coupling bumps, and thereby, entry of water into the joint portions including the coupling bumps may be prevented.

Therefore, the ultrasonic device 120 using flip-chip packaging and having intended characteristics may be provided.

What is claimed is:

1. An ultrasonic device comprising:
a first substrate including a first surface on which a piezoelectric element and a first electrode coupled to the piezoelectric element are placed;
a second substrate including a second surface on which a second electrode coupled to a control circuit is placed;
an intermediate substrate placed between the first substrate and the second substrate and including a third surface joined to the first surface and a fourth surface facing the second surface; and
a bonding portion bonding the second substrate and the intermediate substrate, wherein
the intermediate substrate has a through hole penetrating from the third surface to the fourth surface and a third electrode provided in the through hole and coupled to the first electrode,
the second electrode is coupled to the third electrode and electrically coupled to the first electrode via the third electrode,
a plurality of the bonding portions are provided in island shapes apart from one another between the second substrate and the intermediate substrate, and
at least one of the bonding portions is provided to surround the third electrode.

2. The ultrasonic device according to claim 1, wherein the piezoelectric element is provided in contact with a vibrating film,
an element array area in which a plurality of the piezoelectric elements is arranged is provided, and
the bonding portions are provided in parts not overlapping with the element array area in a plan view.

3. The ultrasonic device according to claim 2, wherein
the intermediate substrate has a rectangular shape in the plan view,
the element array area is provided so that a center of the intermediate substrate is placed inside the element array area, and
the bonding portions are provided in at least three positions apart in a periphery of the element array area.

4. The ultrasonic device according to claim 1, wherein
the third electrode is a bump formed using a resin adhesive agent containing metal filler.

5. The ultrasonic device according to claim 1, wherein
the bonding portion is an insulating resin adhesive agent.

6. The ultrasonic device according to claim 1, wherein
a part of the bonding portion is provided in a peripheral edge part of the intermediate substrate.

7. An ultrasonic diagnostic apparatus comprising the ultrasonic device according to claim 1.

8. An ultrasonic diagnostic apparatus comprising the ultrasonic device according to claim 2.

9. An ultrasonic diagnostic apparatus comprising the ultrasonic device according to claim 3.

10. An ultrasonic diagnostic apparatus comprising the ultrasonic device according to claim 4.

11. An ultrasonic diagnostic apparatus comprising the ultrasonic device according to claim 5.

12. An ultrasonic diagnostic apparatus comprising the ultrasonic device according to claim 6.
